(19) United States
(12) Patent Application Publication
(10) Pub. No.: US 2010/0198186 A1
(43) Pub. Date: Aug. 5, 2010

(54) DUAL-LUMEN CATHETER FOR MEDICAL DEVICE DELIVERY SYSTEMS

(76) Inventor: Simon ACKERMANN, (US)

Correspondence Address: BAKER BOTTS L.L.P., 30 ROCKEFELLER PLAZA, 44TH FLOOR, NEW YORK, NY, 10112-4498, US

(21) Appl. No.: 12/343,622

(22) Filed: Feb. 3, 2009

(51) Int. Cl. A61M 25/14; A61B 6/00

(52) U.S. Cl. 604/500; 600/585; 604/96.01; 604/99.04

(57) ABSTRACT

A catheter assembly having a dual lumen proximal tubular portion including a guidewire lumen and an inflation lumen and a single lumen distal tubular portion that receives the guidewire and can additionally receive fluid. The catheter assembly includes flow passages in allow fluid to flow from the interior of the catheter to the exterior.

### FIELD OF THE INVENTION

The present invention relates to catheters for medical device delivery systems. Particularly, the present invention is directed to dual-lumen catheters capable of flushing contrast medium through a plurality of apertures while a guidewire is positioned in the catheter assembly.

### BACKGROUND OF THE INVENTION

A variety of catheter devices are known in the art for treating the lumenal system of a patient. Of such devices, many are directed to treating the cardiovascular system of a patient.

“Over the wire” or OTW catheters are generally known in the art. These devices are generally introduced into a patient after a guidewire has been introduced into the patient, and advanced to a treatment site within a patient where a treatment procedure (e.g., angioplasty and/or stent placement) is to be performed. A guidewire is initially introduced to the treatment site, a catheter is advanced over the guidewire to the treatment site, the treatment procedure is performed, and the catheter and guidewire are subsequently removed. However, because the guidewire lumen of an over the wire catheter must traverse the entire length of the catheter (which can exceed about 150 cm), either an extremely long guidewire (greater than 300 cm in length) or a guidewire extension must be used to permit the physician to maintain a grip on the guidewire and catheter during the treatment procedure.

To address the problems associated with OTW catheters, an alternative catheter configuration, known as a rapid exchange catheter has been developed. Generally, a rapid exchange catheter has a relatively short guidewire lumen (e.g., about 30 cm) near the distal end of the catheter, thus permitting the physician to use a standard length guidewire (e.g., 150-175 cm) to introduce a catheter and/or perform a catheter exchange.

During vascular therapy such as an angioplasty procedure or the stenting of a lesion site, it is highly desirable to be able to inject a contrast medium such as a radiopaque dye into the vessel upstream of the lesion site in order to check the flow past the site. This enables the physician to precisely locate the stenosis and assist in properly positioning the treatment device prior to treatment. After treatment, the injection of dye allows a determination to be made as to whether the procedure was successful or whether further treatment of the site, manipulation of the stent, or some other procedure, is necessary.

A number of different techniques have previously been employed to deliver contrast medium to a treatment site. In one such method, contrast medium is injected via the guide or guiding catheter and more particularly through the annulus defined between the interior surface of the guide catheter and the exterior surface of the treating catheter extending therethrough. A number of shortcomings are, however, inherent in such approach. Because the guide catheter is relatively large and stiff, its access to smaller arteries and typically those being treated, may be precluded. In the treatment of a coronary artery, for example, the guide catheter can be advanced no further than into the aortic root adjacent the artery being treated. The treatment catheter then extends therefrom into the treatment artery and on to the treatment site. As a consequence, to provide a sufficient concentration of dye to create a satisfactory image past the treatment site, it is necessary to inject a relatively large quantity of the contrast medium. The quantity of dye must be sufficient to fill not only the entire volume of the treatment vessel proximal to the treatment site but must additionally compensate for the significant quantity that can be expected to leak into the aorta as no positive seal is formed between the distal end of the guide catheter and the entrance to the treatment vessel. Moreover, such a large quantity may need to be injected at multiple times or continuously for extended periods of time. However, such repeated injections of large quantities of contrast dye are neither desirable nor cost effective. Delivery of the large quantity of dye that is needed for such a method is further complicated by the fact that the cross-sectional area of the annulus through which the dye is forced along the entire length of the catheter is relatively small. As a result, injection under high pressure is needed to overcome flow restrictions associated with this small cross-sectional injection area.

An alternative to the use of the guide catheter to deliver contrast medium is the use of the guide wire lumen that is formed in the delivery catheter. After a standard over-the-wire balloon catheter or stent deployment catheter is advanced to the treatment site, the guide wire can be removed and the catheter lumen used as a conduit for injection. This minimizes the amount of dye that is needed to generate an image of the treatment site and obviates the possibility of leakage into the aorta thus precluding the distribution of dye to other parts of the body. The principal disadvantage inherent in the use of such technique is that the guide wire must be removed. Because the guide wire must be replaced before another device can be inserted, in case of an emergency, such as a dislodged embolic particle, vessel spasm, or abrupt vessel closure, additional therapeutic devices cannot be positioned quickly. For example, should plaque become dislodged by the injection, the stent would not yet be in position for expansion. Additionally, should such technique be used in conjunction with the delivery of a balloon expandable stent, the treatment catheter must be retracted for each viewing of the site, which increases associated time and risks of the procedures. Moreover, attempting to precisely reposition the catheter in the treatment site after each injection may be difficult to achieve as well.

Such conventional methods and systems generally have been considered satisfactory for their intended purpose. However, a device and associated method is desired to minimize the amount of dye that must be injected into the vasculature for visualization of the treatment site and obviate the need to shift any device within the artery in order to inject the dye.

### SUMMARY OF THE INVENTION

The purpose and advantages of the present invention will be set forth in and apparent from the description that follows, as well as will be learned by practice of the invention. Additional advantages of the invention will be realized and attained by the methods and systems particularly pointed out in the written description and claims hereof, as well as from the appended drawings.

The invention provides a catheter having a flexible shaft having a proximal portion and a distal portion, the shaft having an inflation lumen and a guidewire lumen defined there. The guidewire lumen extends at least along the distal portion of the flexible shaft and has a proximal guidewire port and a distal guidewire port to receive a guidewire therethrough. The flexible shaft further includes at least one flow passage disposed at a location between the proximal guidewire port and the distal guidewire port, the flow passage extending between the guidewire lumen and an outer surface of the flexible shaft. The catheter includes an inflatable member disposed at the distal portion of the flexible shaft and in fluid communication with the inflation lumen, the inflatable member having a proximal end and a distal end. The catheter further includes an inflation adaptor disposed at the proximal portion of the flexible shaft and in fluid communication with the inflatable member via the inflation lumen, and a fluid agent adaptor disposed at the proximal portion of the flexible shaft and in fluid communication with the at least one flow passage via the guidewire lumen.

The flexible shaft of the catheter can include a plurality of flow passages disposed between the proximal guidewire port and the distal guidewire port. The at least one flow passage can be disposed proximal to the inflatable member. The at least one flow passage can be disposed distal to the inflatable member. The flexible shaft can include a tip distal to the inflatable member, the at least one flow passage disposed in the tip. The at least one flow passage can have a cross-sectional dimension less than half a cross-sectional dimension of the proximal guidewire port. The at least one flow passage can include a one-way valve to inhibit fluid flow therethrough into the guidewire lumen. The proximal guidewire port can include a seal to inhibit flow of fluid therethrough from inside the guidewire lumen. The distal guidewire port can be located at a distal end of the flexible shaft and the proximal guidewire port can be located at a proximal end of the flexible shaft with the guidewire lumen extending continuously therebetween. The catheter can include an inner member disposed in the guidewire lumen, the inner member defining an inner guidewire lumen to receive a guidewire therethrough, and a flow lumen co-extensive with the inner guidewire lumen, the fluid agent adaptor in fluid communication with the at least one flow passage via the flow lumen.

In accordance with another aspect of the present invention, a catheter assembly is provided including a guidewire having an outer diameter, and a catheter. The catheter includes a flexible shaft having a proximal portion and a distal portion, the shaft having an inflation lumen and a guidewire lumen defined therein. The guidewire lumen extends at least along the distal portion of the flexible shaft and has a proximal guidewire port and a distal guidewire port to receive the guidewire therethrough. The guidewire lumen has a cross-dimension sufficiently greater than the outer diameter of the guidewire to permit fluid flow therebetween. The catheter includes an inflatable member disposed at the distal portion of the flexible shaft and in fluid communication with the inflation lumen, the inflatable member having a proximal end and a distal end. The catheter includes an inflation adaptor disposed at the proximal portion of the flexible shaft and in fluid communication with the inflatable member via the inflation lumen. The catheter includes a fluid agent adaptor disposed at the proximal portion of the flexible shaft and in fluid communication with the guidewire lumen.

The catheter assembly can include at least one flow passage disposed at a location between the proximal guidewire port and the distal guidewire port, the flow passage extending between the guidewire lumen and an outer surface of the flexible shaft and having a cross-sectional dimension less than the outer diameter of the guidewire. The flexible shaft can include a plurality of flow passages disposed between the proximal guidewire port and the distal guidewire port. The at least one flow passage can be disposed proximal to the inflatable member. The at least one flow passage can be disposed distal to the inflatable member. The at least one flow passage can include a one-way valve to inhibit fluid flow therethrough into the guidewire lumen. The proximal guidewire port can include a seal to engage the guidewire when disposed within the guidewire lumen to inhibit flow of fluid therethrough from inside the guidewire lumen. The distal guidewire port can include a one-way valve to engage the guidewire when disposed within the guidewire lumen to permit fluid flow therethrough from inside the guidewire lumen and inhibit fluid flow therethrough into the guidewire lumen. The distal guidewire port can be located at a distal end of the flexible shaft and the proximal guidewire port can be located at a proximal end of the flexible shaft with the guidewire lumen extending continuously therebetween. The catheter assembly can include an inner member disposed in the guidewire lumen, the inner member defining an inner guidewire lumen to receive a guidewire therethrough, and a flow lumen co-extensive with the inner guidewire lumen, the fluid agent adaptor in fluid communication with the at least one flow passage via the flow lumen. The catheter assembly can include a fluid source coupled with the fluid agent adaptor, the fluid source containing fluid selected from a group consisting of contrast agent, therapeutic agent, diagnostic agent, or medicament.

In accordance with another aspect of the present invention, a method of using a catheter assembly is provided including placing a guidewire having an outer diameter in an intrabody lumen and providing a catheter. The catheter including a flexible shaft having a proximal portion and a distal portion, the shaft having an inflation lumen and a guidewire lumen defined therein, the guidewire lumen extending at least along the distal portion of the flexible shaft and having a proximal guidewire port and a distal guidewire port to receive the guidewire therethrough, the guidewire lumen having a cross-dimension sufficiently greater than the outer diameter of the guidewire to permit fluid flow therebetween. The catheter includes an inflatable member disposed at the distal portion of the flexible shaft and in fluid communication with the inflation lumen, the inflatable member having a proximal end and a distal end. The catheter includes an inflation adaptor disposed at the proximal portion of the flexible shaft and in fluid communication with the inflatable member via the inflation lumen. The catheter includes a fluid agent adaptor disposed at the proximal portion of the flexible shaft and in fluid communication with the guidewire lumen. The method of using a catheter includes positioning the catheter over the guidewire with the guidewire extending through the guidewire lumen and beyond the distal guidewire port and the proximal port, and introducing a fluid through the fluid agent adaptor for release into the intrabody lumen via the guidewire lumen.

The method can include a flexible shaft having at least one flow passage disposed at a location between the proximal guidewire port and the distal guidewire port, the flow passage extending between the guidewire lumen and an outer surface of the flexible shaft and having a cross-sectional dimension less than the outer diameter of the guidewire, the fluid being released into the intrabody lumen through the flow passage. The flexible shaft can include a plurality of flow passages disposed between the proximal guidewire port and the distal guidewire port, the fluid being released into the intrabody lumen through the plurality of flow passages. The at least one flow passage can be disposed proximal to the inflatable member. The at least one flow passage can be disposed distal to the inflatable member. The at least one flow passage can include a one-way valve to inhibit fluid flow therethrough into the guidewire lumen. The proximal guidewire port can include a seal to engage the guidewire when disposed within the guidewire lumen to inhibit flow of fluid therethrough from inside the guidewire lumen. The distal guidewire port can include a one-way valve to engage the guidewire when introducing the fluid to permit the fluid to be released through the distal guidewire port. The catheter can include an inner member disposed in the guidewire lumen, the inner member defining an inner guidewire lumen to receive a guidewire therethrough, and a flow lumen co-extensive with the inner guidewire lumen, the fluid being released into the intrabody lumen through the flow passage. The fluid can be selected from a group consisting of contrast agent, therapeutic agent, diagnostic agent, or medicament.

It is to be understood that both the foregoing general description and the following detailed description are exemplary and are intended to provide further explanation of the invention claimed.

The accompanying drawings, which is incorporated in and constitutes part of this specification, is included to illustrate and provide a further understanding of the method and system of the invention. Together with the description, the drawing serves to explain the principles of the invention.

### BRIEF DESCRIPTION OF THE DRAWINGS

FIG. 1 is a cross sectional side view of a catheter assembly according to an aspect of the present invention.

FIG. 2a is a cross sectional end view of a catheter assembly according to an aspect of the present invention.

FIG. 2b is a cross sectional end view of a catheter assembly according to an aspect of the present invention.

FIG. 3a is a cross-sectional end view of a catheter assembly according to an aspect of the present invention.

FIG. 3b is a cross-sectional end view of a catheter assembly according to an aspect of the present invention.

FIG. 4 is a cross-sectional side view of a catheter assembly according to an aspect of the present invention.

FIG. 5 is a cross-sectional side view of a catheter assembly according to an aspect of the present invention.

FIG. 6 is a cross-sectional side view of a catheter assembly according to an aspect of the present invention.

FIG. 7a is a cross-sectional end view of a catheter assembly according to an aspect of the present invention.

FIG. 7b is a cross-sectional end view of a catheter assembly according to an aspect of the present invention.

FIG. 8a is a cross-sectional end view of a catheter assembly according to an aspect of the present invention.

FIG. 8b is a cross-sectional end view of a catheter assembly according to an aspect of the present invention.

FIG. 9 is a cross-sectional side view of a catheter assembly according to an aspect of the present invention.

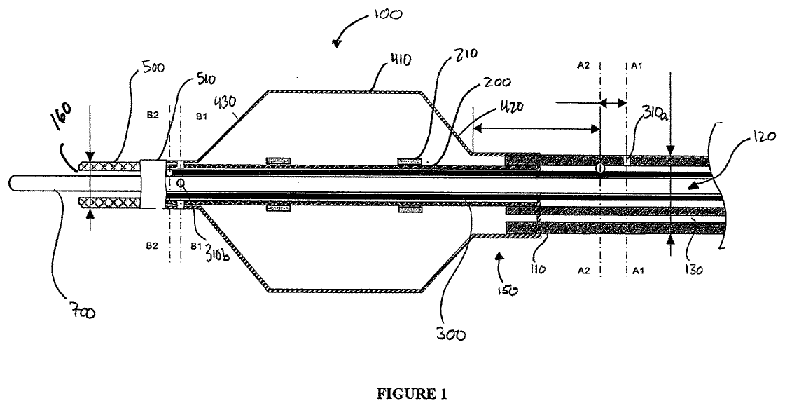

While the invention is capable of various modifications and alternative forms, specific embodiments thereof have been shown by way FIGS. 1-9, and will herein be described in detail. It should be understood, however, that it is not intended to limit the invention to the particular forms disclosed but, on the contrary, the intention is to cover all modifications, equivalents, and alternatives falling within the spirit and scope of the invention as defined by the appended claims.

### DETAILED DESCRIPTION OF THE EMBODIMENTS

Reference will now be made in detail to the embodiments of the invention, an example of which is illustrated in the accompanying drawings.

The use of the terms “a” and “an” and “the” and similar terms in the context of describing the invention are to be construed to cover both the singular and the plural, unless otherwise indicated herein or clearly contradicted by context.

The terms “body lumen,” “vascular wall,” and “vascular lumen” are to be construed as open-ended terms (i.e., meaning “including, but not limited to”) unless otherwise noted. These terms are all interpreted to be target tissues, and to further include an “body cavity.” The medical device as described herein can be utilized by one skilled in the art for treating any suitable condition.

The terms “comprising,” “having,” “including,” and “containing” are to be construed as open-ended terms (i.e., meaning “including, but not limited to”) unless otherwise noted.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced in ways other than those specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

For purpose of explanation and illustration, and not limitation, an exemplary embodiment of the catheter assembly in accordance with the invention is shown in FIG. 1 and is designated generally by reference character 100.

As shown in FIG. 1, the catheter assembly 100 generally includes a flexible shaft 150 including a proximal tubular portion 110 and a distal tubular portion 200. The flexible shaft 150 includes at least two lumen therein. For example, as depicted in FIG. 1, the proximal portion 110 has a guidewire lumen 120 and an inflation lumen 130 therein. Guidewire lumen 120 and inflation lumen 130 can be arranged in a side-by-side configuration.

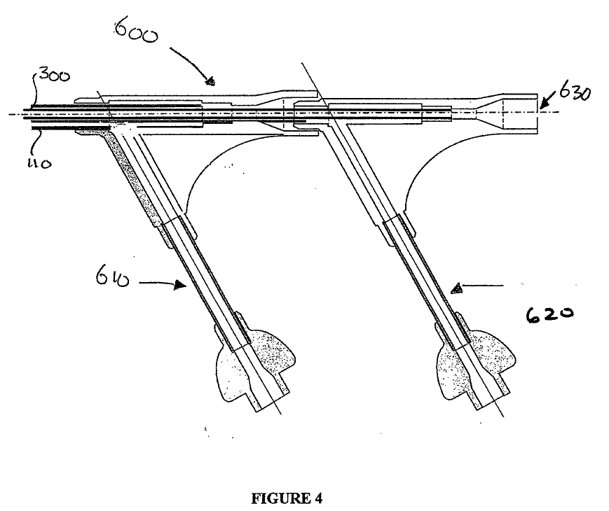

As shown in FIG. 1, the catheter assembly includes an inflatable member 410 disposed at the distal portion 200 of the flexible shaft 150. Inflatable member 410 is in fluid communication with inflation lumen 130. Inflatable member 410 has proximal end 420 and distal end 430. The catheter assembly includes a proximal guidewire port 630 (shown in FIG. 4) and a distal guidewire port 160 to receive guidewire 700 therethrough.

In accordance with one aspect of the present invention, inner tubular member 300 is disposed within the lumen of the distal tubular member 200 and the guidewire lumen 120 of the proximal tubular member 110. Inner tubular member 300 is configured to have a size that accommodates a guide wire 700. In such a configuration, fluid such as but not limited to contrast agent can be injected at fluid agent adaptor 620 and into guidewire lumen 120. The fluid, in this example, contrast agent, is received through guidewire lumen 120 of proximal portion 110, and through distal portion 200. The fluid is received between an outer surface of inner tubular member 300 and within guidewire lumen 120 in the proximal portion and distal portion 200 in the distal portion. The fluid is pushed out of catheter assembly 100 through flow passages 310.

Flexible shaft 150 includes at least one flow passage, shown generally as flow passage 310, disposed at a location between the proximal guidewire port 630 and the distal guidewire port 160. Flow passage 310 extends from the guidewire lumen to an outer surface of flexible shaft 150. In one embodiment, at least one flow passage 310 is disposed near the proximal end of balloon 410.

As generally depicted in FIG. 4, catheter assembly 100 can further include a manifold 600 at the proximal end of catheter assembly 100. As shown, manifold 600 can include an inflation adaptor 610 and fluid agent adaptor 620. FIG. 4 shows inflation adaptor 610 disposed at the proximal portion 110 of flexible shaft 150. Inflation adaptor 610 is in fluid communication with inflatable member 410 via inflation lumen 130. Inflation adaptor 610 is used to push inflation fluid through inflation lumen 130 in order to inflate inflatable member 410. FIG. 4 shown the catheter assembly including a fluid agent adaptor 620 disposed at the proximal portion of flexible shaft 150. Fluid agent adaptor 620 is in fluid communication with the flow passages 310 via the guidewire lumen 120. Fluid agent adaptor 620 can flush fluids such as contrast agent, therapeutic agent, diagnostic agent, or medicament through guidewire lumen 120 and out flow passages 310 to treat or visualize a treatment site.

The catheter assembly 100, as shown in FIG. 1 can include a plurality of flow passages 310, disposed between the proximal guidewire port 630 and the distal guidewire port 160. Flow passages 310 can be disposed proximal to inflatable member 410, as shown by flow passage 310a. Flow passages 310 can also be disposed distal to inflatable member 410, as shown by flow passage 310b.

The catheter assembly 100 of the present invention can include a tip member 500 distal to inflatable member 410. Tip member 500 can act as a flow passage 310. Tip member 500 can be configured to have a taper, such as a distal taper. Tip member 500 is secured to the distal end of distal portion 200, for example, by joint 510.

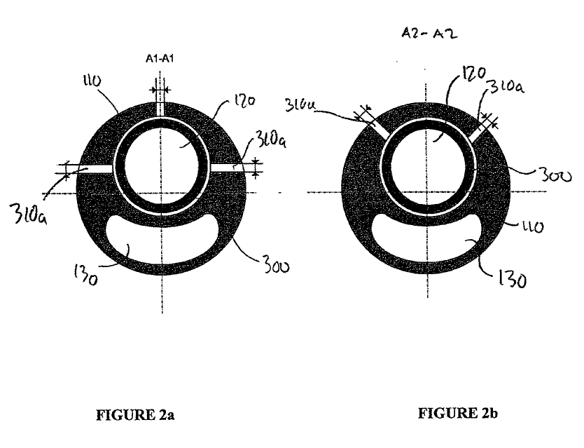

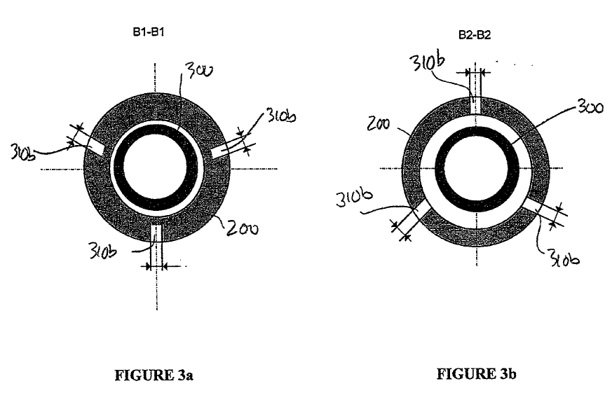

As shown in FIGS. 2a, 2b, 3a, and 3b, catheter assemblies 100 in accordance with the present invention can include a variety of configurations of flow passages 310. FIGS. 2a and 2b show cross-sectional views of catheter assembly 100 along lines A1 and A2, respectively. Inner tubular member 300, which can extend a part or the entire length of catheter assembly 100 is located within guidewire lumen 120. FIGS. 2a and 2b illustrate two different configurations of proximal flow passages 310a. Flow passages 310a are in fluid communication with guidewire lumen 120 and as shown may be positioned at any desired angle or location along flexible shaft 150 proximal to inflatable member 410. FIGS. 3a and 3b show cross-sectional views of catheter assembly 100 along lines B1 and B2, respectively. As shown in FIGS. 3a and 3b, inner tubular member 300 is located within guidewire lumen 120 of distal member 200, and distal flow passages 310b extend from an outer surface of distal member 200 to guidewire lumen 120. A catheter assembly in accordance with the present invention may include any suitable number or configuration of proximal and distal flow passages 310.

The flow passages 310 can be disposed in a linear arrangement or spatially arranged. At least one distal flow passage 310b may be disposed approximately 10 mm from the distal end 430 of inflatable member 410. Flow passages 310 are configured to have a diameter (size) to permit passage of fluid from the interior of the catheter assembly to the exterior of the catheter assembly. For example and not limitation, the diameter of apertures 310 may be about less than 0.1 mm in diameter.

In accordance with one aspect of the present invention, flow passages 310 may be located on both proximal portion 110 and distal portion 200 to permit the passage of fluid to the exterior of the catheter assembly. If however, both the distal and proximal portions, 110 and 200, respectively, include apertures, it is preferable that the flow passages 310a on proximal portion 110 have a larger diameter than flow passages 310b on distal portion 200 to compensate for pressure differential.

As shown in FIG. 1, distal tubular portion 200 can include one or more radiopaque markers 210. The radiopaque marker(s) 210 can be secured to the inner or outer surfaces of distal tubular portion 200. For example and not limitation, radiopaque marker 210 can be secured by heat bonding, adhering with glue or other adhesive, or swaged into the tubular member.

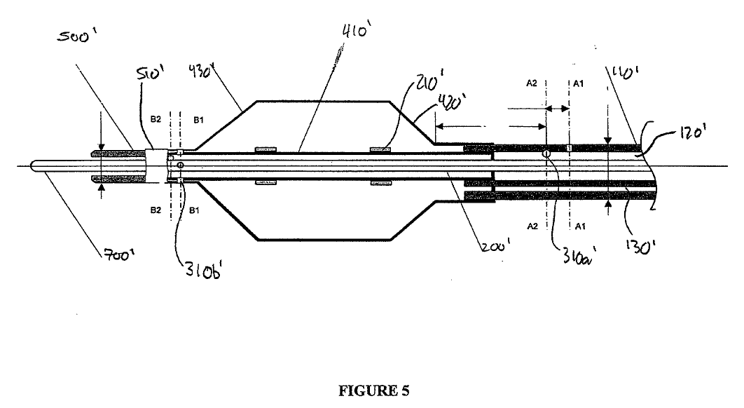

In accordance with one embodiment of the present invention shown in FIGS. 5-9. As shown in FIG. 5, the catheter assembly 100′ generally includes a flexible shaft 150′ including a proximal tubular portion 110′ and a distal tubular portion 200′. The flexible shaft 150′ includes at least two lumen therein. For example, as depicted in FIG. 5, the proximal portion 110′ has a guidewire lumen 120′ and an inflation lumen 130′ therein. Guidewire lumen 120′ and inflation lumen 130′ can be arranged in a side-by-side configuration.

As shown in FIG. 5, the catheter assembly includes an inflatable member 410′ disposed at the distal portion 200′ of the flexible shaft 150′. Inflatable member 410′ is in fluid communication with inflation lumen 130′. Inflatable member 410′ has proximal end 420′ and distal end 430′. The catheter assembly includes a proximal guidewire port 630′ and a distal guidewire port 160′ to receive guidewire 700′ therethrough.

The aspect of the present invention depicted in FIGS. 5-9 does not include an inner tubular member. In such a configuration, guidewire lumen 120′ in the proximal portion 110′ and the distal portion 200′ is sized to receive both a guidewire 700′ and a fluid such as contrast agent (not shown). The fluid, in this example, contrast agent, is received through guidewire lumen 120′ of proximal portion 110′, and through distal portion 200′. The fluid is received within guidewire lumen 120′ in the proximal portion and distal portion 200′ in the distal portion. The fluid is pushed out of catheter assembly 100′ through flow passages 310′.

Flexible shaft 150′ includes at least one flow passage, shown generally as flow passage 310′, disposed at a location between the proximal guidewire port 630′ and the distal guidewire port 160′. Flow passage 310′ extends from the guidewire lumen to an outer surface of flexible shaft 150′.

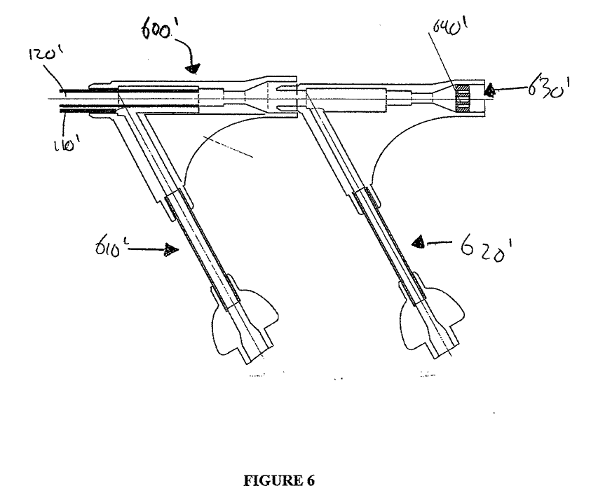

As generally depicted in FIG. 6, catheter assembly 100′ can further include a manifold 600′ at the proximal end of catheter assembly 100′. As shown, manifold 600′ can include an inflation adaptor 610′ and fluid agent adaptor 620′. FIG. 6 shows inflation adaptor 610′ disposed at the proximal portion 110′ of flexible shaft 150′. Inflation adaptor 610′ is in fluid communication with inflatable member 410′ via inflation lumen 130′. Inflation adaptor 610′ is used to push inflation fluid through inflation lumen 130′ in order to inflate inflatable member 410′. FIG. 6 shown the catheter assembly including a fluid agent adaptor 620′ disposed at the proximal portion of flexible shaft 150′. Fluid agent adaptor 620′ is in fluid communication with the flow passages 310′ via the guidewire lumen 120′. Fluid agent adaptor 620′ can flush fluids such as contrast agent, therapeutic agent, diagnostic agent, or medicament through guidewire lumen 120′ and out flow passages 310′ to treat or visualize a treatment site. As shown in FIG. 6, the manifold 600′ can include an additional sealing member 640′ to prevent fluid from escaping through proximal guidewire port 630′.

The catheter assembly 100′, as shown in FIG. 5 can include a plurality of flow passages 310′, disposed between the proximal guidewire port 630′ and the distal guidewire port 160′. Flow passages 310′ can be disposed proximal to inflatable member 410′, as shown by flow passage 310a′. Flow passages 310′ can also be disposed distal to inflatable member 410, as shown by flow passage 310b′.

The catheter assembly 100′ can include a tip member 500′ distal to inflatable member 410′. Tip member 500′ can act as a flow passage 310′. Tip member 500′ can be configured to have a taper, such as but not limited to a distal taper. Tip member 500′ is secured to the distal end of distal portion 200′, for example, by joint 510′.

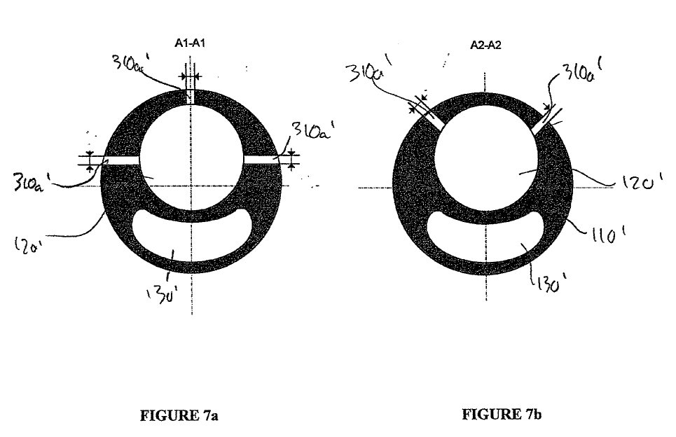

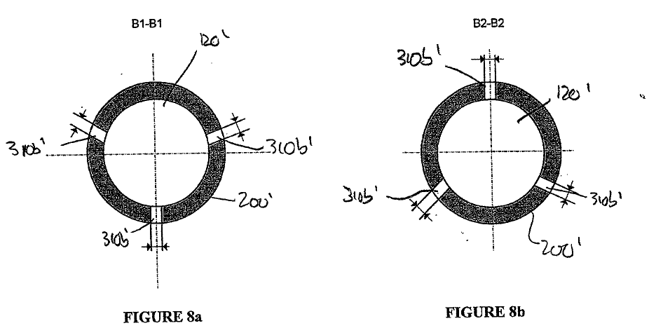

As shown in FIGS. 7a, 7b, 8a, and 8b, catheter assemblies 100′ in accordance with the present invention can include a variety of configurations of flow passages 310′. FIGS. 7a and 7b show cross-sectional views of catheter assembly 100′ along lines A1 and A2, respectively. FIGS. 7a and 7b illustrate two different configurations of proximal flow passages 310a′. Flow passages 310a′ are in fluid communication with guidewire lumen 120′ and as shown may be positioned at any desired angle or location along flexible shaft 150′ proximal to inflatable member 410′. FIGS. 8a and 8b show cross-sectional views of catheter assembly 100′ along lines B1 and B2, respectively. As shown in FIGS. 8a and 8b, distal flow passages 310b′ extend from an outer surface of distal member 200′ to guidewire lumen 120. A catheter assembly in accordance with the present invention may include any suitable number or configuration of proximal and distal flow passages 310′.

The flow passages 310′ can be disposed in a linear arrangement or spatially arranged. At least one distal flow passage 310b′ may be disposed approximately 10 mm from the distal end 430′ of inflatable member 410′. Flow passages 310′ are configured to have a diameter (size) to permit passage of fluid from the interior of the catheter assembly to the exterior of the catheter assembly. For example and not limitation, the diameter of apertures 310′ may be about less than 0.1 mm in diameter.

In accordance with one aspect of the present invention, flow passages 310′ may be located on both proximal portion 110′ and distal portion 200′ to permit the passage of fluid to the exterior of the catheter assembly. If however, both the distal and proximal portions, 110′ and 200′, respectively, include apertures, it is preferable that the flow passages 310a′ on proximal portion 10′ have a larger diameter than flow passages 310b′ on distal portion 200′ to compensate for pressure differential.

As shown in FIG. 5, distal tubular portion 200′ can include one or more radiopaque markers 210′. The radiopaque marker(s) 210′ can be secured to the inner or outer surfaces of distal tubular portion 200′. For example and not limitation, radiopaque marker 210′ can be secured by heat bonding, adhering with glue or other adhesive, or swaged into the tubular member.

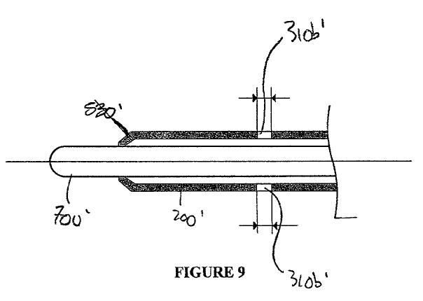

FIG. 9 shows one embodiment of tip member 500′ that includes a tapered portion 530′ that tapers down to seal against guidewire 700′.

The tubular members of the embodiments can be formed from any conventional material or blends, as would be known in the art. Preferably, the tubular members are formed of polymers having sufficient flexibility to traverse the vasculature of a patient. A non-limiting list of materials for example, include polyamides, such as any nylon, polyimides, polyesters, such as polyethylene terephthalate, block copolymers such as Pebax® or Hytrel®, or a combination or blend thereof. Preferably, the tip member is formed of a material that is softer than that of the distal tubular member, i.e., preferably the tip member has a lower durometer than at least the proximal shaft member.

It will be apparent to those skilled in the art that various modifications and variations can be made in the method and system of the present invention without departing from the spirit or scope of the invention. Thus, it is intended that the present invention include modifications and variations that are within the scope of the appended claims and their equivalents.

What is claimed is:

1. A catheter comprising:
a flexible shaft having a proximal portion and a distal portion, the shaft having an inflation lumen and a guidewire lumen defined therein, the guidewire lumen extending at least along the distal portion of the flexible shaft and having a proximal guidewire port and a distal guidewire port to receive a guidewire therethrough, the flexible shaft further including at least one flow passage disposed at a location between the proximal guidewire port and the distal guidewire port, the flow passage extending between the guidewire lumen and an outer surface of the flexible shaft;
an inflatable member disposed at the distal portion of the flexible shaft and in fluid communication with the inflation lumen, the inflatable member having a proximal end and a distal end;
an inflation adaptor disposed at the proximal portion of the flexible shaft and in fluid communication with the inflatable member via the inflation lumen; and
a fluid agent adaptor disposed at the proximal portion of the flexible shaft and in fluid communication with the at least one flow passage via the guidewire lumen.

2. The catheter of claim 1, wherein the flexible shaft includes a plurality of flow passages disposed between the proximal guidewire port and the distal guidewire port.

3. The catheter of claim 1, wherein the at least one flow passage is disposed proximal to the inflatable member.

4. The catheter of claim 1, wherein the at least one flow passage is disposed distal to the inflatable member.

5. The catheter of claim 1, wherein the shaft includes a tip distal to the inflatable member, the at least one flow passage is disposed in the tip.

6. The catheter of claim 1, wherein the at least one flow passage has a cross dimension less than half a cross dimension of the proximal guidewire port.

7. The catheter of claim 1, wherein the at least one flow passage includes a one-way valve to inhibit fluid flow therethrough into the guidewire lumen.

8. The catheter of claim 1, wherein the proximal guidewire port includes a seal to inhibit flow of fluid therethrough from inside the guidewire lumen.

9. The catheter of claim 1, wherein the distal guidewire port is located at a distal end of the flexible shaft and the proximal guidewire port is located at a proximal end of the flexible shaft with the guidewire lumen extending continuously therebetween.

10. The catheter of claim 1, further comprising an inner member disposed in the guidewire lumen, the inner member defining an inner guidewire lumen to receive a guidewire therethrough and a flow lumen co-extensive with the inner guidewire lumen, the fluid agent adaptor in fluid communication with the at least one flow passage via the flow lumen.

11. A catheter assembly comprising:
a guidewire having an outer diameter; and
a catheter including:
a flexible shaft having a proximal portion and a distal portion, the shaft having an inflation lumen and a guidewire lumen defined therein, the guidewire lumen extending at least along the distal portion of the flexible shaft and having a proximal guidewire port and a distal guidewire port to receive the guidewire therethrough, the guidewire lumen having an cross-dimension sufficiently greater than the outer diameter of the guidewire to permit fluid flow therebetween,
an inflatable member disposed at the distal portion of the flexible shaft and in fluid communication with the inflation lumen, the inflatable member having a proximal end and a distal end,
an inflation adaptor disposed at the proximal portion of the flexible shaft and in fluid communication with the inflatable member via the inflation lumen, and
a fluid agent adaptor disposed at the proximal portion of the flexible shaft and in fluid communication with the guidewire lumen.

12. The catheter assembly of claim 11, wherein the flexible shaft includes at least one flow passage disposed at a location between the proximal guidewire port and the distal guidewire port, the flow passage extending between the guidewire lumen and an outer surface of the flexible shaft and having a cross-sectional dimension less than the outer diameter of the guidewire.

13. The catheter assembly of claim 12, wherein the flexible shaft includes a plurality of flow passages disposed between the proximal guidewire port and the distal guidewire port.

14. The catheter assembly of claim 12, wherein the at least one flow passage is disposed proximal to the inflatable member.

15. The catheter assembly of claim 12, wherein the at least one flow passage is disposed distal to the inflatable member.

16. The catheter assembly of claim 12, wherein the at least one flow passage includes a one-way valve to inhibit fluid flow therethrough into the guidewire lumen.

17. The catheter assembly of claim 11, wherein the proximal guidewire port includes a seal to engage the guidewire when disposed within the guidewire lumen to inhibit flow of fluid therethrough from inside the guidewire lumen.

18. The catheter assembly of claim 11, wherein the distal guidewire port includes a one-way valve to engage the guidewire when disposed within the guidewire lumen to permit fluid flow therethrough from inside the guidewire lumen and inhibit fluid flow therethrough into the guidewire lumen.

19. The catheter assembly of claim 11, wherein the distal guidewire port is located at a distal end of the flexible shaft and the proximal guidewire port is located at a proximal end of the flexible shaft with the guidewire lumen extending continuously therebetween.

20. The catheter assembly of claim 11, further comprising an inner member disposed in the guidewire lumen, the inner member defining an inner guidewire lumen to receive a guidewire therethrough and a flow lumen co-extensive with the inner guidewire lumen, the fluid agent adaptor in fluid communication with the flow lumen.

21. The catheter assembly of claim 11, further comprising a fluid source coupled with the fluid agent adaptor, the fluid source containing fluid selected from a group consisting of contrast agent, therapeutic agent, diagnostic agent or medicament.

22. A method of using a catheter assembly comprising:
placing a guidewire having an outer diameter in an intrabody lumen;
providing a catheter including:
a flexible shaft having a proximal portion and a distal portion, the shaft having an inflation lumen and a guidewire lumen defined therein, the guidewire lumen extending at least along the distal portion of the flexible shaft and having a proximal guidewire port and a distal guidewire port to receive the guidewire therethrough, the guidewire lumen having an cross-dimension sufficiently greater than the outer diameter of the guidewire to permit fluid flow therebetween,
an inflatable member disposed at the distal portion of the flexible shaft and in fluid communication with the inflation lumen, the inflatable member having a proximal end and a distal end,
an inflation adaptor disposed at the proximal portion of the flexible shaft and in fluid communication with the inflatable member via the inflation lumen, and
a fluid agent adaptor disposed at the proximal portion of the flexible shaft and in fluid communication with the guidewire lumen;
positioning the catheter over the guidewire with the guidewire extending through the guidewire lumen and beyond the distal guidewire port and the proximal port;
introducing a fluid through the fluid agent adaptor for release into the intrabody lumen via the guidewire lumen.

23. The method of claim 22, wherein the flexible shaft includes at least one flow passage disposed at a location between the proximal guidewire port and the distal guidewire port, the flow passage extending between the guidewire lumen and an outer surface of the flexible shaft and having a cross-sectional dimension less than the outer diameter of the guidewire, the fluid being released into the intrabody lumen through the flow passage.

24. The method of claim 23, wherein the flexible shaft includes a plurality of flow passages disposed between the proximal guidewire port and the distal guidewire port, the fluid being released into the intrabody lumen through the plurality of flow passages.

25. The method of claim 23, wherein the at least one flow passage is disposed proximal to the inflatable member, the fluid being released into the intrabody lumen proximal to the inflatable member.

26. The method of claim 23, wherein the at least one flow passage is disposed distal to the inflatable member, the fluid being released into the intrabody lumen distal to the inflatable member.

27. The method of claim 22, wherein the proximal guidewire port includes a seal, the seal engaging the guidewire when introducing the fluid to inhibit the fluid from flowing through the proximal guidewire port.

28. The method of claim 22, wherein the distal guidewire port includes a one-way valve to engage the guidewire when introducing the fluid to permit the fluid to be released through the distal guidewire port.

29. The method of claim 22, wherein the catheter further comprises an inner member disposed in the guidewire lumen, the inner member defining an inner guidewire lumen to receive a guidewire therethrough and a flow lumen co-extensive with the inner guidewire lumen, the fluid being released into the intrabody lumen via the flow lumen.

30. The method of claim 22, wherein the fluid is selected from a group consisting of contrast agent, therapeutic agent, diagnostic agent or medicament.
